(19) United States
(12) Patent Application Publication
(10) Pub. No.: US 2010/0069712 A1
(43) Pub. Date: Mar. 18, 2010

(54) MOTHER-BABY ENDOSCOPE

(75) Inventor: Koji Yamaya, Tokyo (JP)

Correspondence Address: SCULLY SCOTT MURPHY & PRESSER, PC, 400 GARDEN CITY PLAZA, SUITE 300, GARDEN CITY, NY, 11530, US

(73) Assignee: OLYMPUS MEDICAL SYSTEMS CORP., Tokyo (JP)

(21) Appl. No.: 12/557,860

(22) Filed: Sep. 11, 2009

(30) Foreign Application Priority Data
Sep. 12, 2008 (JP) 2008-234980

(51) Int. Cl. A61B 1/018; A61B 1/00

(52) U.S. Cl. 600/107; 600/113

(57) ABSTRACT

A mother-baby endoscope includes: a lateral-view mother endoscope housing a treatment instrument raising stand in a distal end portion and including a treatment instrument insertion channel disposed in an insertion portion and opening toward the treatment instrument raising stand; and a baby endoscope including an insertion portion having a bending portion disposed on a distal end side, the insertion portion being inserted into the treatment instrument insertion channel of the mother endoscope and guided into a body cavity for observing or treating a region to be inspected. When the insertion portion of the baby endoscope is protruded by approximately 20 millimeters from an observation window of the mother endoscope in an observation direction, a proximal end surface of the bending portion of the baby endoscope is set to be located on a more proximal end side than an opening portion of the treatment instrument insertion channel.

This application claims the benefit of Japanese Application No. 2008-234980 filed in Japan on Sep. 12, 2008, the contents of which are incorporated herein by this reference.

### BACKGROUND OF THE INVENTION

1. Field of the Invention

The present invention relates to a mother-baby endoscope, and more particularly to a mother-baby endoscope capable of observing or treating the inside of a bile duct or a pancreatic duct by inserting a baby endoscope, which is inserted through a treatment instrument insertion channel of a mother endoscope, from a duodenal papilla selectively into the bile duct or the pancreatic duct.

2. Description of the Related Art

Conventionally, what is called a mother-baby endoscope has been put into practical use as an endoscope which is used when observing or treating the inside of the bile duct or the pancreatic duct.

This mother-baby endoscope has a normal-sized endoscope as a mother endoscope, for example, a duodenum endoscope, and an endoscope having a thin diameter as a baby endoscope. The baby endoscope is inserted through a treatment instrument insertion channel of the mother endoscope and protruded from the distal end of the mother endoscope into a body cavity. By performing various operations in combination at the operation portions on the hand side of a user, for example, bending operation, advancing/retracting operation, and twisting operation of the baby endoscope, and raising stand operation, bending operation, twisting operation, advancing/retracting operation, and the like, of the mother endoscope, only the baby endoscope is inserted from the duodenal papilla selectively into the bile duct or the pancreatic duct to observe or treat the inside of the bile duct or the pancreatic duct.

Various types of conventional mother-baby endoscopes have been proposed, for example, in Japanese Patent Application Laid-Open Publication No. 2007-75168, and put into practical use.

The mother-baby endoscope disclosed in the Japanese Patent Application Laid-Open Publication No. 2007-75168, for example, includes in the insertion portion of the mother endoscope a treatment instrument insertion channel through which the insertion portion of the baby endoscope can be inserted. The mother-baby endoscope also includes inside the distal end portion of the mother endoscope a treatment instrument raising stand as a mechanism which works on the insertion portion of the baby endoscope protruded from the opening portion of the treatment instrument insertion channel and can change the advancing/retracting direction of the distal end portion of the baby endoscope by raising the insertion portion of the baby endoscope in the vicinity of the opening portion of the treatment instrument insertion channel.

### SUMMARY OF THE INVENTION

A mother-baby endoscope according to the present invention includes: a lateral-view mother endoscope housing a treatment instrument raising stand in a distal end portion of the mother endoscope and including a treatment instrument insertion channel disposed in an insertion portion of the mother endoscope, the treatment instrument insertion channel being open toward the treatment instrument raising stand; and a baby endoscope including an insertion portion having a bending portion disposed on a distal end side, the insertion portion being adapted to be inserted into the treatment instrument insertion channel of the mother endoscope and guided into a body cavity for observing or treating a region to be inspected, wherein, when the insertion portion of the baby endoscope is protruded by approximately 20 millimeters from an observation window of the mother endoscope in an observation direction, a proximal end surface of the bending portion of the baby endoscope is located on a more proximal end side than an opening portion of the treatment instrument insertion channel of the mother endoscope.

Furthermore, a mother-baby endoscope according to the present invention includes: a lateral-view mother endoscope housing a treatment instrument raising stand in a distal end portion of the mother endoscope and including a treatment instrument insertion channel disposed in an insertion portion of the mother endoscope, the treatment instrument insertion channel being open toward the treatment instrument raising stand; and a baby endoscope including an insertion portion having a bending portion disposed on a distal end side, the insertion portion being adapted to be inserted into the treatment instrument insertion channel of the mother endoscope and guided into a body cavity for observing or treating a region to be inspected, wherein, when the insertion portion of the baby endoscope is protruded by approximately 20 millimeters from an observation window of the mother endoscope in an observation direction, a central axis on a more proximal end side than the bending portion of the insertion portion of the baby endoscope positioned in the treatment instrument insertion channel becomes substantially linear.

The advantages of the present invention will be more apparent from the detailed description given below.

### BRIEF DESCRIPTION OF THE DRAWINGS

FIG. 1 is an exterior view schematically showing a state where a baby endoscope is inserted into a mother endoscope in a mother-baby endoscope according to an embodiment of the present invention.

FIG. 2 is a main-part enlarged cross-sectional view showing in an enlarged manner a vicinity of a distal end portion of the mother endoscope of the mother-baby endoscope in the state shown in FIG. 1.

FIG. 3 is a main-part enlarged cross-sectional view showing in an enlarged manner a distal end portion main body of a mother endoscope according to a first modified example of the treatment instrument insertion channel of the mother endoscope of the embodiment of the present invention.

FIG. 4 is a front view of a distal end side of a ferrule member opening portion of a treatment instrument insertion channel of the mother endoscope in FIG. 3.

FIG. 5 is a front view of the distal end side of the ferrule member opening portion similarly to FIG. 4, showing a second modified example of the treatment instrument insertion channel of the mother endoscope according to the embodiment of the present invention.

FIG. 6 is a view showing a case where a normal detachable distal end cover is mounted to a distal end portion main body of the mother endoscope of the mother-baby endoscope according to the embodiment of the present invention.

FIG. 7 is a view showing a case where a detachable distal end cover of a third modified example is mounted to the distal end portion main body of the mother endoscope of the mother-baby endoscope according to the embodiment of the present invention.

FIG. 8 is a view showing a case where a detachable distal end cover of a fourth modified example is mounted to the distal end portion main body of the mother endoscope of the mother-baby endoscope according to the embodiment of the present invention.

FIG. 9 is a schematic perspective view of a distal end hood to be mounted to the distal end portion main body of the mother endoscope of the mother-baby endoscope according to the embodiment of the present invention.

FIG. 10 is a view showing a screen displaying an endoscopic image when the mother endoscope to which the distal end hood in FIG. 9 is mounted is inserted in a large intestine.

### DETAILED DESCRIPTION OF THE PREFERRED EMBODIMENTS

First, a schematic configuration of a mother-baby endoscope of the present embodiment will be described below with reference to the drawings.

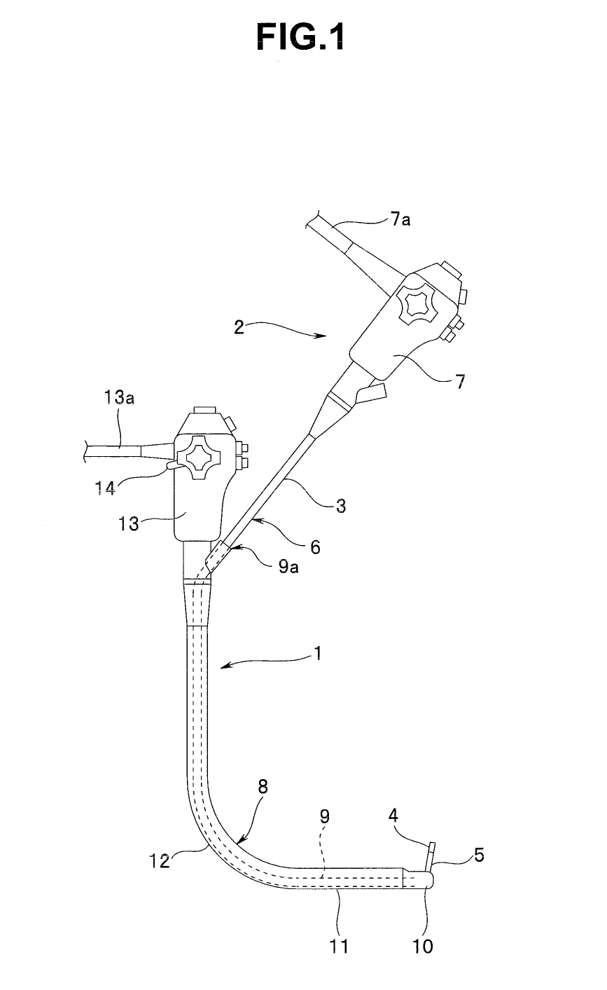

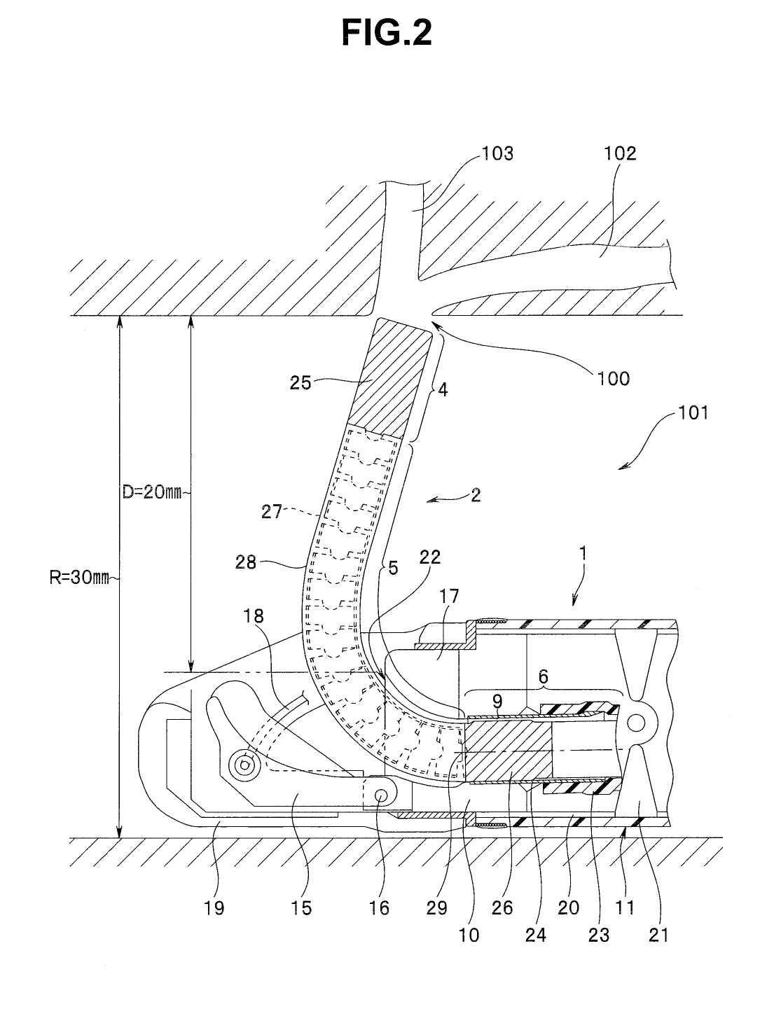

FIGS. 1 and 2 are views showing the mother-baby endoscope of the embodiment of the present invention. FIG. 1 is an exterior view schematically showing a state where a baby endoscope is inserted in a mother endoscope according to the embodiment of the present invention. FIG. 2 is a main-part enlarged cross-sectional view showing in an enlarged manner a vicinity of a distal end portion of the mother endoscope of the mother-baby endoscope in the state shown in FIG. 1.

The mother-baby endoscope of the present embodiment is configured of a mother endoscope 1 and a baby endoscope 2.

The mother endoscope 1 is configured of an elongated-shaped insertion portion 8 and an operation portion 13 provided in a linked manner on a proximal end side of the insertion portion 8.

The insertion portion 8 is formed to have an outer diameter of approximately 10 millimeters and a length of approximately 1.2 meters. The insertion portion 8 is configured by including: a distal end portion main body 10; a bending portion 11; and a flexible tube portion 12, which are provided in a linked manner in this order from the distal end side.

A treatment instrument insertion channel 9 is formed inside the insertion portion 8. The treatment instrument insertion channel 9 passes from an opening 9a provided to the operation portion 13 to the distal end portion main body 10. The treatment instrument insertion channel 9 is formed to allow passage therethrough of an insertion portion 3 of the baby endoscope 2, for example, in addition to a treatment instrument and the like.

Inside the distal end portion main body 10 is provided a treatment instrument raising stand 15 (not shown in FIG. 1, see FIG. 2) for raising the treatment instrument or the insertion portion 3 of the endoscope 2, which is inserted through the treatment instrument insertion channel 9 and protruded from a distal end opening 22 (see FIG. 2) of the treatment instrument insertion channel 9.

The operation portion 13 is configured by including various operation members required for operating the mother endoscope 1, a universal cable 13a for connecting between the operation portion 13 and an endoscope unit (not shown) for controlling the mother endoscope 1, and the like.

Provided as one of the operation members of the operation portion 13, for example, is an operation lever 14 for operating the action of the above-mentioned treatment instrument raising stand 15.

On the other hand, the baby endoscope 2 is configured of the elongated-shaped insertion portion 3 and an operation portion 7 provided in a linked manner on a proximal end side of the insertion portion 3.

The insertion portion 3 is formed to have an outer diameter of approximately 3 to 4 millimeters and a length of approximately 2 meters. The insertion portion 3 is configured by including: a distal end portion main body 4 in which an observation window, an illumination window (not shown) and the like are arranged; a bending portion 5; and a flexible tube portion 6, which are provided in a linked manner in this order from the distal end side.

The operation portion 7 is configured by including various operation members required for operating the baby endoscope 2, a universal cable 7a for connecting the operation portion 7 and an endoscope unit (not shown) for controlling the baby endoscope 2, and the like.

Inside the distal end portion main body 10 of the mother endoscope 1, as shown in FIG. 2, the treatment instrument raising stand 15 is disposed in the vicinity of the distal end opening 22 of the treatment instrument insertion channel 9. The treatment instrument raising stand 15 is rotatably disposed centering around a spindle 16 supported at a fixed region (not shown) of the distal end portion main body 10. In addition, to the treatment instrument raising stand 15 is joined one end portion of an operation wire 18 extended from the operation portion 13 (see FIG. 1) and inserted and arranged in the insertion portion 8. The other end of the operation wire 18 is joined to the operation lever 14 (see FIG. 1) of the operation portion 13.

According to this configuration, when the operation lever 14 is operated, the operation wire 18 is advanced and retracted in an axial direction in the insertion portion 8. The advancing/retracting movement of the operation wire 18 rotates the treatment instrument raising stand 15 centering around the spindle 16. Accordingly, this operation enables the treatment instrument raising stand 15 to tilt or rise.

In a state where the insertion portion 3 of the baby endoscope 2 is inserted through the treatment instrument insertion channel 9 and the insertion portion 3 is protruded from the distal end opening 22 of the treatment instrument insertion channel 9, if the treatment instrument raising stand 15 is operated as described above, the treatment instrument raising stand 15 works on the insertion portion 3 of the baby endoscope 2 and can change the protruding direction of the insertion portion 3.

Note that an electric insulation member 17 is disposed in the vicinity of the distal end opening 22 of the treatment instrument insertion channel 9 so as to enable insulation of the channel, even if an electrode of a high-frequency treatment instrument (not shown) contacts the channel, for example. However, the electric insulation member 17 is not directly related to the gist of the present invention, so that it is not an essential configuration to dispose the electric insulation member 17.

Outer side of the distal end portion main body 10 of the mother endoscope 1 is covered with a distal end cover 19. In addition, on the proximal end side of the distal end portion main body 10 is provided in a linked manner a bending tube 21 configured by joining a plurality of bending pieces 20.

The treatment instrument insertion channel 9 is mainly configured of a channel tube 23, a ferrule member 24 for joining the channel tube 23 to the distal end portion main body 10, and the distal end portion main body 10 including the electric insulation member 17.

On the other hand, in the baby endoscope 2, the distal end side and the proximal end side of the bending portion 5 are provided with rigid portions 25 and 26 (see the regions shown by diagonal lines in FIG. 2), respectively. The rigid portion 25 on the distal end side is the distal end portion main body 4. Furthermore, the rigid portion 26 on the proximal end side configures a part of the flexible tube portion 6, and includes, for example, a joining member for joining the flexible tube portion 6 and a bending portion 5.

The bending portion 5 is configured by joining a plurality of bending pieces 27 (shown by dotted lines in FIG. 2) one another through joint shafts (not shown). The outer surface side of the bending portion 5 is covered with a bending rubber 28. The bending rubber 28 is made of, for example, fluorine-based rubber, urethane-based rubber, elastomer, and the like. However, there is no limitation placed on the kind of the material as long as the material has elasticity and biocompatibility.

Note that a proximal end surface 29 of the bending portion 5 shown in FIG. 2 is defined as a position of the last joint shaft disposed on the proximal-most side among the components of the bending portion 5.

Next, description will be made on the working when using the mother-baby endoscope thus configured according to the present embodiment.

Generally, it is said that the inner diameter of the lumen of the duodenum of the human being is approximately around 30 millimeters in usual cases (see reference numeral R in FIG. 2). On the other hand, the inner diameter of the lateral-view endoscope as the mother endoscope, for example, the duodenum endoscope is set to approximately around 10 millimeters in usual cases.

Considering these facts, the distance from the orifice of the duodenal papilla 100 to the observation window of the mother endoscope is derived to be approximately 20 millimeters (see reference numeral D in FIG. 2).

As shown in FIG. 2, description will be made on the case where the mother-baby endoscope of the present embodiment is inserted into a lumen 101 of the duodenum and the baby endoscope 2 is inserted into a bile duct 102 or a pancreatic duct 103 to perform observation or treatment.

FIG. 2 shows an operational situation of extracting the baby endoscope 2 from the bile duct or the pancreatic duct.

That is, FIG. 2 specifically shows that the treatment instrument raising stand 15 of the mother endoscope 1 is tilted and the baby endoscope 2 is pulled out from the proximal end side thereof, and the state at the moment that the distal end of the baby endoscope 2 has been pulled out from the orifice of the duodenal papilla 100.

At this time, in the present embodiment, the proximal end surface 29 of the bending portion 5 of the baby endoscope 2 is located at a position on the more proximal end side than the distal end opening 22 of the treatment instrument insertion channel 9 of the mother endoscope 1. Therefore, the distal end side of the flexible tube portion 6 of the baby endoscope 2 is substantially linear. Accordingly, the restoring force for linearizing the flexible tube portion 6 when the flexible tube portion 6 is more or less in a bent state is not generated. Note that the bending portion 5 is kept in the bent state due to the working of the bending pieces 27.

Even if the distal end portion main body 4 of the baby endoscope 2 is extracted from the duodenal papilla 100 in such a state, the shape of the baby endoscope 2 is not changed before and after the extraction. Therefore, such a configuration is free from the conventionally encountered problem, that is, the distal end portion main body 4 or bending portion 5 of the baby endoscope 2 moves swiftly due to the restoring force of the flexible tube portion 6.

Therefore, such a configuration prevents the distal end portion main body 4 or bending portion 5 of the baby endoscope 2 from bumping against and thus damaging the treatment instrument raising stand 15 and its peripheral components.

As described above, according to the present embodiment, when the distal end of the baby endoscope 2 is protruded by approximately 20 millimeters from the observation window of the mother endoscope 1 in the observation direction (see FIG. 2), the proximal end surface 29 of the bending portion 5 of the baby endoscope 2 is located at a position on the more proximal end than the distal end opening 22 of the treatment instrument insertion channel 9 of the mother endoscope 1. This brings about the state where no restoring force is generated at the part (the flexible tube portion 6) on the more proximal end side than the bending portion 5 of the baby endoscope 2, that is, the flexible tube portion 6 becomes substantially linear.

Accordingly, when the baby endoscope 2 is extracted from the duodenal papilla 100, the distal end side of the baby endoscope 2 does not move, which prevents the bending portion 5 of the baby endoscope 2 from bumping against and damaging the treatment instrument raising stand 15 and its peripheral components.

Next various modified examples of the mother-baby endoscope according to the embodiment of the present invention will be disclosed below.

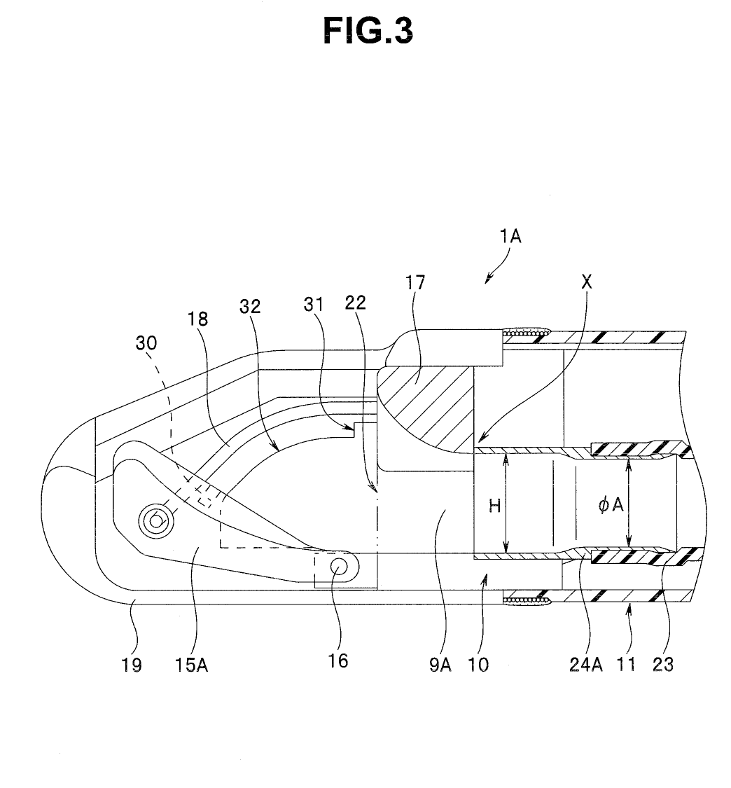

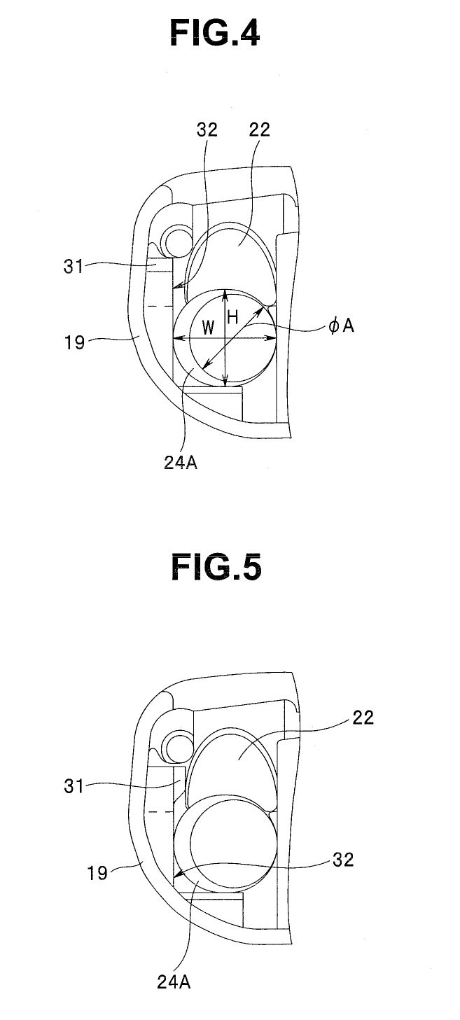

First, a first modified example of the treatment instrument insertion channel of the mother endoscope of the above-described embodiment will be described with reference to FIGS. 3 and 4. FIG. 3 is a main-part enlarged cross-sectional view showing in an enlarged manner the distal end portion main body of the mother endoscope according to the above-described embodiment. FIG. 4 is a front view of a distal end side of the ferrule member opening portion of the treatment instrument insertion channel.

A ferrule member 24A of a mother endoscope 1A according to the first modified example is formed such that the inner diameter H on the distal end side has a larger opening diameter than that of the inner diameter φA n the proximal end side ((φA<H).

As shown in FIG. 4, when viewing the opening portion of the ferrule member 24A from the distal end side thereof, it is seen that the opening portion is extended larger than the proximal end side not only in the up/down direction (the vertical direction in the drawing) but also in the left/right direction (lateral direction in the figure). That is, the opening diameter of the treatment instrument insertion channel 9A is set to satisfy both φA<H and φA<W.

In addition, in order to prevent as much as possible damaging the bending rubber of the baby endoscope (not shown in FIG. 3) inserted through the treatment instrument insertion channel 9A, the distal end part of the ferrule member 24 is formed to have a reduced number of minimal level differences created at borders between the distal end part and other parts. The distal end of the ferrule member 24A is fixed by adhesive or the like to the distal end portion main body 10 with the distal end in contact with the rear surface of the electric insulation member 17 (see the region shown by the reference numeral X in FIG. 3).

Note that the ferrule member 24A is made of metal member and formed such that the inner shape, i.e., the inner diameter thereof smoothly varies in the substantially central part.

Furthermore, to prevent damaging the bending rubber of the baby endoscope (not shown) inserted through the treatment instrument insertion channel 9, the inner and outer surfaces of the ferrule member 24A are subjected to surface finishing to remove its edge portions using a barrel grinding stone or by magnetic polishing, for example.

On the other hand, a treatment instrument raising stand 1 5A has on its side surface a projection portion 30 which is shown by dotted lines in FIG. 3. The projection portion 30 comes into contact with a raising restriction surface 31 of the distal end portion main body 10 when the treatment instrument raising stand 15A is raised to the maximum. That is, the maximum raising position of the treatment instrument raising stand 15A is defined by bringing the projection portion 30 of the treatment instrument raising stand 15A into contact with the raising restriction surface 31.

According to such a configuration, when seeing from the front face as shown in FIG. 4, the raising restriction surface 31 is disposed on a sliding surface 32 of the treatment instrument raising stand 15A without protruding from the sliding surface 32, so that the bending rubber of the baby endoscope is not damaged by the raising-restriction surface 31 even when the baby endoscope is inserted through the treatment instrument insertion channel 9A.

Next, FIG. 5 is a view showing a second modified example in which the raising restriction surface 31 is disposed at a position different from that in the first modified example shown in FIG. 4.

As shown in FIG. 5, the raising restriction surface 31 is arranged on the inner side than the sliding surface 32.

The raising restriction surface 31, however, is arranged at a position not contacting the bending rubber of the baby endoscope when the baby endoscope is inserted through the treatment instrument insertion channel 9A.

This configuration thus also prevents damaging the bending rubber of the baby endoscope.

Next, various examples of the mother endoscope in the case where the detachable distal end cover is mounted to the mother endoscope will be disclosed.

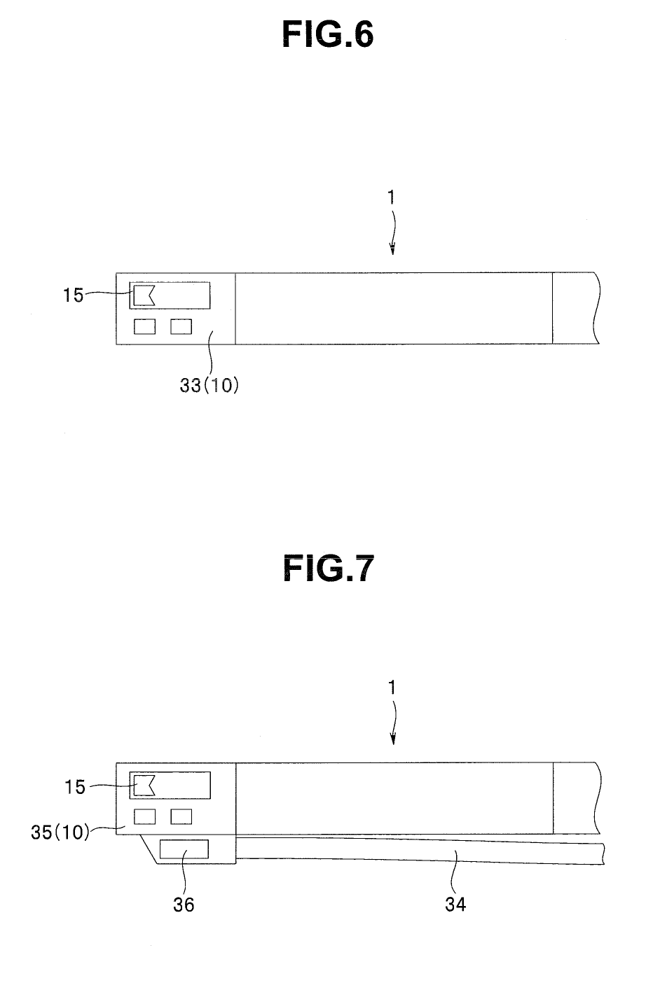

The example shown in FIG. 6 shows a case where a normal detachable distal end cover is mounted to the distal end portion main body of the mother endoscope.

FIG. 7 shows an exemplary case where a detachable distal end cover according to a third modified example is mounted to the mother endoscope of the mother-baby endoscope of the present invention.

As shown in FIG. 7, a detachable distal end cover 35 of the present modified example is arranged so as to cover the distal end portion main body 10 of the mother endoscope 1.

The detachable distal end cover 35 is a detachable distal end cover with an external channel, which is configured by adding a second channel 34 to the normal detachable distal end cover 33 shown in FIG. 6.

Similarly as the normal treatment instrument insertion channel, the second channel 34 is formed to allow passage therethrough of the treatment instrument and the baby endoscope. In addition, a second channel opening portion 36 is formed in the vicinity of the distal end portion of the second channel 34.

Accordingly, a user can easily and surely obtain two channels by mounting the detachable distal end cover 35 in FIG. 7, instead of the detachable distal end cover 33 in FIG. 6, as the distal end cover to be mounted to the mother endoscope 1 in accordance with a desired treating method for each case.

Note that, in the example shown in FIG. 7, the treatment instrument raising stand is not provided inside the second channel opening portion 36. However the example is not limited to this configuration. The treatment instrument raising stand may be provided inside the second channel opening portion 36.

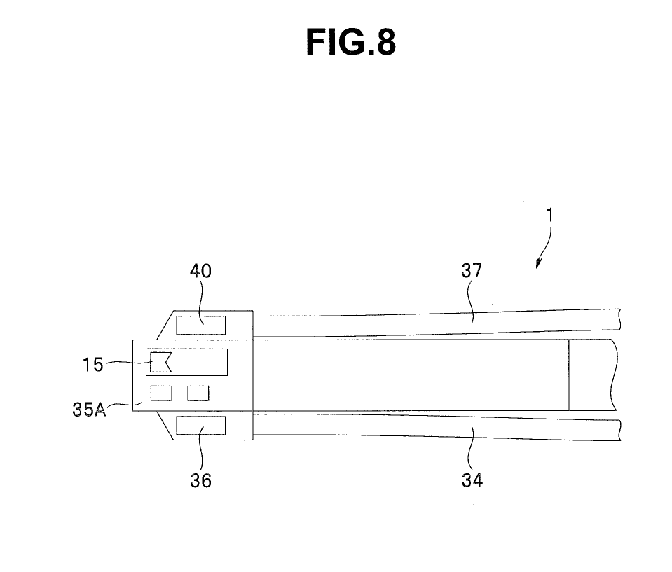

FIG. 8 shows an exemplary case where the detachable distal end cover according to a fourth modified example is mounted to the mother endoscope of the mother-baby endoscope of the present invention.

As shown in FIG. 8, the detachable distal end cover 35A of the present modified example is a detachable distal end cover with external channels, which is configured by further adding a third channel 37 to the detachable distal end cover 35 according to the above-described modified example.

Similarly as the normal treatment instrument insertion channel and the second channel 34, the third channel 37 is formed to allow passage therethrough of the treatment instrument and the baby endoscope. In addition, a third channel opening portion 40 is formed in the vicinity of the distal end portion of the third channel 37. Accordingly, a user can easily and surely obtain three channels by mounting the detachable distal end cover 35A in FIG. 8, instead of the detachable distal end cover in FIG. 6 and the detachable distal end cover 35 in FIG. 7, as the distal end cover to be mounted to the mother endoscope 1 in accordance with a desired treating method for each case.

Next, an exemplary configuration of the distal end hood will be disclosed.

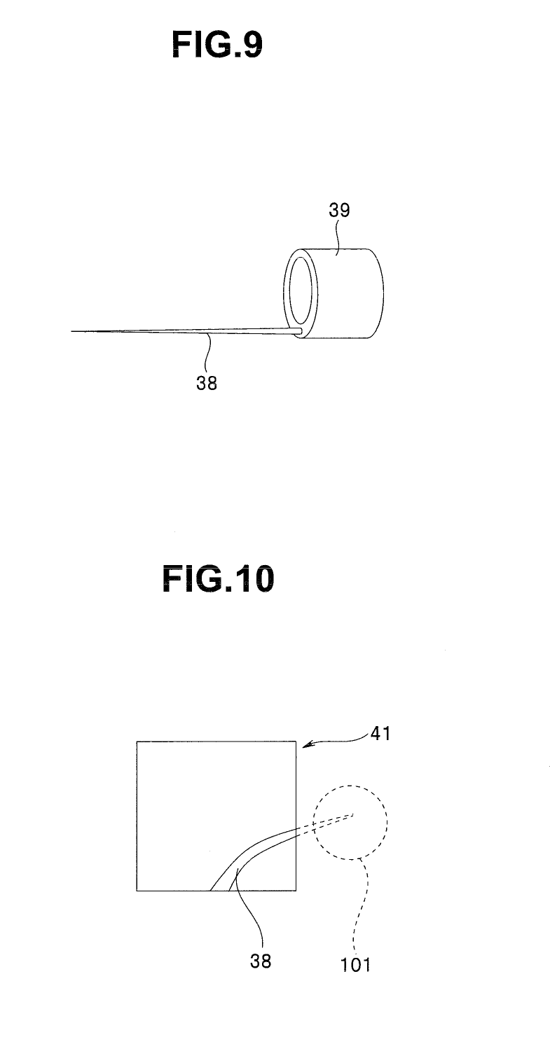

FIG. 9 is a schematic perspective view of the distal end hood of the present exemplary configuration. In addition, FIG. 10 is a view showing a screen displaying an endoscopic image when the distal end hood in FIG. 9 is mounted to the mother endoscope and the mother endoscope is inserted into a large intestine.

As shown in FIG. 9, a distal end hood 39 of the present example is configured by including a guide wire 38 having a length of approximately 20 centimeters.

Since the guide wire 38 generally has a straight-advancing property, the guide wire 38 indicates the deep direction of the lumen even when the lumen 101 is outside the field of view of the endoscopic image and is not observable.

According to this configuration, as shown in FIG. 10, if a part of the guide wire 38 is displayed within the frame of a screen 41 as an endoscopic image, it is possible to estimate that the distal end of the guide wire 38 exists on the extended line of the endoscopic image. This facilitates finding the lumen, thus advantageously reducing the inspection time.

Note that it is needless to say that the present invention is not limited to the above-described embodiment, and various changes, modifications, and applications are possible without departing from the gist of the invention. Furthermore, the above-described embodiment includes inventions of various stages, and by combining a plurality of constituent components disclosed in the embodiment, inventions of various stages can also be extracted. For example, even if some constituent components are deleted from all the constituent components shown in the above-described present embodiment, if the problem described in the section of BACKGROUND OF THE INVENTION can be solved and the effects described in the section of SUMMARY OF THE INVENTION can be obtained, the configuration in which some constituent components are deleted can be extracted as an invention. The present invention is not limited by specific embodiments but is defined by appended claims.

What is claimed is:

1. A mother-baby endoscope comprising:
a lateral-view mother endoscope housing a treatment instrument raising stand in a distal end portion of the mother endoscope and including a treatment instrument insertion channel disposed in an insertion portion of the mother endoscope, the treatment instrument insertion channel being open toward the treatment instrument raising stand; and
a baby endoscope including an insertion portion having a bending portion disposed on a distal end side, the insertion portion being adapted to be inserted into the treatment instrument insertion channel of the mother endoscope and guided into a body cavity for observing or treating a region to be inspected,
wherein, when the insertion portion of the baby endoscope is protruded by approximately 20 millimeters from an observation window of the mother endoscope in an observation direction, a proximal end surface of the bending portion of the baby endoscope is located on a more proximal end side than an opening portion of the treatment instrument insertion channel of the mother endoscope.

2. A mother-baby endoscope comprising:
a lateral-view mother endoscope housing a treatment instrument raising stand in a distal end portion of the mother endoscope and including a treatment instrument insertion channel disposed in an insertion portion of the mother endoscope, the treatment instrument insertion channel being open toward the treatment instrument raising stand; and
a baby endoscope including an insertion portion having a bending portion disposed on a distal end side, the insertion portion being adapted to be inserted into the treatment instrument insertion channel of the mother endoscope and guided into a body cavity for observing or treating a region to be inspected,
wherein, when the insertion portion of the baby endoscope is protruded by approximately 20 millimeters from an observation window of the mother endoscope in an observation direction, a central axis on a more proximal end side than the bending portion of the insertion portion of the baby endoscope positioned in the treatment instrument insertion channel becomes substantially linear.
